(19) United States
(12) Patent Application Publication
(10) Pub. No.: US 2026/0354788 A1
(43) Pub. Date: Nov. 12, 2026

(54) METHOD FOR IMAGING BRAIN VASCULAR ACTIVITY

(71) Applicants: ICONEUS, Antony (FR); INSTITUT NATIONAL DE LA SANTÉ ET DE LA RECHERCHE MÉDICALE, Paris (FR); ECOLE SUPÉRIEURE DE PHYSIQUE ET DE CHIMIE INDUSTRIELLES DE LA VILLE DE PARIS, Paris (FR); CENTRE NATIONAL DE LA RECHERCHE SCIENTIFIQUE, Paris (FR)

(72) Inventors: Bruno-Felix OSMANSKI, Sartrouville (FR); Thomas DEFFIEUX, Villejuif (FR); Mickaël TANTER, Bagneux (FR); Anatole JIMENEZ, Vincennes (FR); Thomas GABEREL, Caen (FR)

(21) Appl. No.: 19/111,899

(22) PCT Filed: Oct. 4, 2023
(86) PCT No.: PCT/EP2023/077393

§ 371 Date: Mar. 14, 2025

(30) Foreign Application Priority Data
Oct. 4, 2022 (EP) 22306482.5

(51) Int. Cl. A61B 8/08; A61B 8/00; A61B 8/12; A61B 90/10

(52) U.S. Cl. CPC A61B 8/0808; A61B 8/0891; A61B 8/12; A61B 8/4254; A61B 8/4461; A61B 8/469; A61B 8/488; A61B 8/5223; A61B 8/5261; A61B 2090/103

(57) ABSTRACT

Traumatic brain injuries and aneurysm-related hemorrhages are significant causes of death and severe disabilities in human and animal subjects. More specifically, brain lesions significantly arise from secondary ischemic damages in comatose subjects. The present disclosure proposes a reliable method for monitoring vascular activity in at least one region of a brain, of a human or animal, the method including obtaining a temporal series of 3D Doppler scans of said brain in said region with an ultrasound probe (4) which has been implanted through a burr hole (BH) in said human or animal, and computing a temporal evolution (21, 22, 23) of at least one vascular parameter of said region based on said 3D Doppler scans of said temporal series.

### TECHNICAL FIELD

This present disclosure concerns methods and apparatuses for imaging brain vascular activity in a human or animal.

### BACKGROUND ART

Traumatic brain injuries and aneurysm-related hemorrhages are significant causes of death and severe disabilities in human and animal subjects. More specifically, brain lesions significantly arise from secondary ischemic damages in comatose subjects.

Maintenance of the comatose subjects' cerebral blood supply is the cornerstone of neurocritical care. Hence, a reliable method for monitoring brain vascular activity, and in particular for imaging early secondary ischemia, is crucial for appropriate neurocritical care.

Unfortunately, existing imaging modalities do not fulfill this task. For instance, transcranial Doppler imaging modalities are limited by a spatial coverage. Hence, transcranial Doppler imaging modalities do not allow to observe brain vascular activity outside the spatial cover, preventing from imaging early secondary ischemia outside the limiting spatial cover. Further, transcranial Doppler imaging modalities are not adapted to monitor brain vascular activity. Likewise, neither computed tomography (CT) perfusion, nor magnetic resonance (MR) perfusion imaging modalities are suitable to monitor brain vascular activity. On the other hand, intracranial pressure monitoring modalities allow to monitor brain vascular activity. Nevertheless, intracranial pressure monitoring modalities do not allow to image early secondary ischemia.

Therefore, there exists a need for reliably monitoring brain vascular activity in a human or animal.

### SUMMARY

To this end, the present disclosure proposes a method for imaging vascular activity in at least one region of a brain, of a human or animal, the method including:
(a) obtaining a temporal series of three-dimensional, 3D, Doppler scans of said brain in said region based on ultrasound measurements of said region performed with an ultrasound probe having an array of at least one ultrasound transducer, the ultrasound probe having been implanted through a burr hole in said human or animal;
(b) computing a temporal evolution of at least one vascular parameter of said region based on said 3D Doppler scans of said temporal series.

The proposed method advantageously allows, depending on the embodiment, to monitor brain vascular activity. Owing to the computed, and even in some embodiments continuous, temporal evolution of the at least one vascular parameter of the region, ischemia, and in particular early secondary ischemia, occurring in the region may be detected.

Notably, in some embodiments, the inventors achieved to image vascular activity in a region covering 80% of a swine brain. That is, ischemia, and in particular early secondary ischemia, occurring anywhere in the four quarters of the swine brain may be detected by a method of the present disclosure.

What is more, in some embodiments, the ultrasound probe may have been implanted through the burr hole for a duration comprised between an hour and several days, allowing to monitor the brain activity for said duration.

The following features, can be optionally implemented, separately or in combination one with the others:

In one or more embodiments, the method further includes:
(a1) obtaining an additional temporal series of additional images of said brain in said region based on additional ultrasound measurements of said region performed with said ultrasound probe,
(b1) computing an additional temporal evolution of at least one additional vascular parameter of said region based on said additional images of said additional temporal series, wherein the additional images are chosen in the group comprising: B-mode ultrasound images, elastography images and any combination thereof.

In some embodiments, the additional temporal series may be acquired in parallel to the temporal series of 3D Doppler scans. That is, for some of the times points, and even in some embodiments for each time point, of the temporal series of 3D Doppler scans, an additional image of said brain in said region may be obtained. The additional images may be 2D or 3D images. Acquiring B-mode ultrasound images may allow to enrich the information regarding the region by computing the additional temporal evolution of at least one additional vascular parameter of said region. The additional vascular parameter may be a ventricle's size of a ventricle in said region. Acquiring elastography images may allow to enrich the information regarding the region by computing the additional temporal evolution of at least one additional vascular parameter of said region. The additional vascular parameter may be an intracranial pressure in said region.

In one or more embodiments, the method further includes:
(c) triggering an alert based on a variation of the temporal evolution of the at least one vascular parameter exceeding a threshold.

Anormal cerebral blood supply's variations may hence be, in some embodiments, automatically detected. For instance, detection of a decrease in the vascular parameter's values may allow to detect an ischemia. Alternatively or in combination, detection of a spike in the temporal evolution of the at least one vascular parameter may allow to detect a blood clot's migration preceding the blockage of a vessel and the resulting ischemia.

In one or more embodiments, the triggering of the alert is based on a duration of the variation of the temporal evolution of the at least one vascular parameter exceeding the threshold.

For instance, the alert may be triggered when the variation of the temporal evolution of the at least one vascular parameter exceeds the threshold for a duration comprised between 1 s and 10 h. One example of usable duration is 1 min. Hence, artifactual variations may not trigger an alert, reinforcing the reliability of the method.

In one or more embodiments, the method further includes obtaining a baseline of the at least one vascular parameter and the variation of the temporal evolution of the at least one vascular parameter depends on the baseline.

The baseline may be chosen as a reference for the at least one vascular parameter in the region. In some embodiments, the variation of the temporal evolution of the at least one vascular parameter may be computed as a relative variation of the vascular parameter's values with respect to the baseline. In some embodiments, the threshold for the variation of the temporal evolution at least one vascular parameter in the region may be defined based on the baseline. For instance, the threshold may be a percentage of relative variation with respect to the baseline. The percentage may be comprised between −100% and +500%, preferably −50%. That is, different thresholds may be set for vascular parameters and/or regions. Further, different thresholds may hence be set for different subjects. Therefore, in some embodiments, different thresholds may be configured for each subject, vascular parameter and region of the brain, allowing to increase the detection sensitivity of the vascular activity's variations.

In one or more embodiments, the method further includes:
(d) obtaining at least a first ultrasound localization microscopy image of said brain in said region based on first additional ultrasound measurements of said region performed with said ultrasound probe.

In one or more embodiments, the 3D Doppler scans may be computed based on ultrafast ultrasound images. Acquiring at least one additional ultrasound localization microscopy image may allow to obtain at least an image of the region with a higher resolution, enriching the information regarding the region. What is more, the at least one additional image may be acquired using the same ultrasound probe as for the 3D Doppler scans acquisition. Hence, the same probe may allow to monitor vascular activity in the region through the 3D Doppler scans, to detect anormal variations and to gain better insight in the activity in the region through higher resolution imaging.

In one or more embodiments, the method further includes, based on the triggered alert:
(e) obtaining at least a second ultrasound localization microscopy image of said brain in said region based on second additional ultrasound measurements of said region performed with said ultrasound probe,
(f) comparing said at least second ultrasound localization microscopy image with said at least first ultrasound localization microscopy image.

Advantageously, an additional baseline may be based on the additional first ULM image allowing to gain precision in the evaluation of the vascular parameter's variation. For instance, the first ULM image may be acquired prior to the acquisition of the temporal series of 3D Doppler scans. That is, the additional baseline with a higher resolution may be acquired prior to the acquisition of the temporal series of 3D Doppler scans. Then, the second ULM image may be acquired based on the triggered alert, in particular when the alert is triggered. Said second ULM image may be compared with said first ULM image. In particular, the value of the at least one vascular parameter of said region in the second ULM image may be compared with the value of the at least one vascular parameter of said region in the first ULM image. Hence, the comparison between the ULM images prior to and posterior to the triggered alert may allow to quantitify the change in the vascular activity due to the triggered alert with a resolution up to micron scale.

In one or more embodiments, the at least one vascular parameter is chosen in the group comprising: blood flow, blood velocity, blood volume, arterial resistivity index and any combination thereof.

Computing a parameter among blood flow, blood velocity, blood volume, arterial resistivity index, and any combination thereof may allow to derive a behavior of the at least one region of the vascular network. The vascular activity of the at least one region may hence be quantified without requiring a large amount of information from different types.

In one or more embodiments, the at least one 3D Doppler scan of said temporal series is registered to at least one of a brain atlas, a computed tomography scan of said brain, a magnetic resonance image scan of said brain and any combination thereof.

Registering the at least one Doppler scan to one of a brain atlas, a CT scan of said brain, a MRI scan of scan and any combination thereof may allow to combine complementary information for a subject, such as anatomical and physiological information. In particular, the brain atlas may be a structural brain atlas, a vascular brain atlas or a functional brain atlas. Registering the at least one 3D Doppler scan to the brain atlas may further allow to compare vascular activity between different subjects, whose burr hole do not always have the same locations.

In one or more embodiments, said temporal series of 3D Doppler scans of said brain in said region is divided into areas of interest, and the method further includes for each area of interest:
(b1) computing a temporal evolution of at least one vascular parameter of said area of interest based on said 3D Doppler scans of said temporal series.

Dividing said region into areas of interest may allow to increase the granularity of the computed temporal evolution. Hence, different behaviors in the areas of interest of the region may be observed. That is, a global vision of the imaged region of the brain may be obtained with additional local information in the areas of interests, increasing the sensitivity of the observations.

In one or more embodiments, the ultrasound is coupled to an accelerometer configured to detect a movement of said human or animal, and the alert is triggered based on said detection of said movement.

Detecting a movement of said human or animal, in particular a movement of said human or animal's head, may allow to distinguish false positive alerts. More precisely, the subject may move or be manipulated, which may temporarily modify the vascular activity in the imaged region. In this case, the modification in the vascular activity may not be pathological and the alert may not be triggered.

In one or more embodiments, the ultrasound probe is rotated between measurements to obtain at least a 3D Doppler scan of said temporal series.

Rotating the ultrasound probe between measurements may allow to obtain the 3D Doppler scan with an ultrasound probe adapted for 2D imaging. Further, the 2D Doppler images acquired at different rotation angles may be obtained with the same pattern of interrogation ultrasonic beams, providing homogeneity in the acquisition of the 3D Doppler scan. Alternatively, a matrix array probe may be used to acquire the 3D Doppler scans.

In one or more embodiments, a pause is observed between two successive rotations of the ultrasound probe.

High power transmission may be needed for deep imaging of the brain region. Further, the ultrasound probe may have a small aperture, in particular since the ultrasound probe is configured to fit in the burr hole. Consequently, a probe's heating safety limit may be surpassed if no pause is observed between two successive rotations of the ultrasound probe. Therefore, implementing a pause between two successive rotations of the ultrasound probe may allow to remain under the probe's heating safety limit.

The present disclosure also concerns an apparatus for imaging vascular activity in at least one region of a brain, of a human or animal, said apparatus including:
(a) an ultrasound measuring device adapted to perform ultrasound measurements of said region with an ultrasound probe having an array of at least one ultrasound transducer, the ultrasound probe being implantable through a burr hole;
(b) a computing module adapted to:
   compute, from said ultrasound measurements, a temporal series of three-dimensional, 3D, Doppler scans of said region;
   compute a temporal evolution of at least one vascular parameter of said region based on said 3D Doppler scans of said temporal series.

In embodiments of the apparatus, the computing module is adapted to detect a seizure and/or a delayed ischemia, vasospam, hemorrhage, hypoperfusion, hyperperfusion or edema based on the temporal evolution of the at least one vascular parameter of said region.

In another aspect, it is proposed a computer software comprising instructions to implement at least a part of a method as defined here when the software is executed by a processor. In another aspect, it is proposed a computer-readable non-transient recording medium on which a software is registered to implement the method as defined here when the software is executed by a processor.

In another aspect, it is proposed a method for imaging vascular activity in at least one region of a brain, of a human or animal, the method comprising:
performing one burr hole into the skull of said human or animal;
implanting through said burr hole an implatable ultrasound probe having an array of at least one ultrasound transducer;
obtaining a temporal series of three-dimensional, 3D, Doppler scans of said brain in said region based on ultrasound measurements of said region performed with said implantable ultrasound probe;
computing a temporal evolution of at least one vascular parameter of said region based on said 3D Doppler scans of said temporal series.

### BRIEF DESCRIPTION OF DRAWINGS

Other features, details and advantages will be shown in the following detailed description and on the figures, on which:

FIG. 1 is a block diagram illustrating an embodiment of an apparatus according to the present disclosure.

FIG. 2 illustrates a possible method of obtaining a series of 3D Doppler scans with the apparatus of FIG. 1.

FIG. 3 illustrates part of the apparatus in use in one specific embodiment.

FIG. 4 illustrates part of the apparatus in use in one specific embodiment.

FIG. 5(a) shows a 2D Doppler image of a region of the brain obtained with a method of the present disclosure.

FIG. 5(b) shows an ULM image of the same region of the brain as in FIG. 5(a), the ULM image being obtained with a method of the present disclosure.

FIG. 5(c) shows a zoom of the ULM image of FIG. 5(b).

FIG. 6(a) shows an example of 3D Doppler scans before and after a stroke, the 3D Doppler scans being obtained with a method of the present disclosure.

FIG. 6(b) shows an example of perfusion CT images before and after a stroke.

FIG. 7(a) shows an example of 2D Doppler images during a stroke establishment, the 2D Doppler images being obtained with a method of the present disclosure.

FIG. 7(b) shows an example of a measure of an evolution of at least one vascular parameter during a stroke establishment, the measure being obtained with a method of the present disclosure.

### DESCRIPTION OF EMBODIMENTS

In the Figures, the same references denote identical or similar elements.

The present disclosure proposes a method and apparatus for imaging vascular activity in at least one region of a brain, of a human or animal, by obtaining a temporal series of three-dimensional, 3D, Doppler scans of said brain in said region. The 3D Doppler scans are obtained with an ultrasound probe, which has been implanted through a burr hole. The temporal series of 3D Doppler scans enables to obtain values of at least one vascular parameter in at least one area of interest of said region. Based on the values of the at least one vascular parameter from the temporal series of 3D Doppler scans, a measure of a temporal evolution of the at least one vascular parameter pay be computed.

The region of the brain may cover between 25% and 100% of the brain.

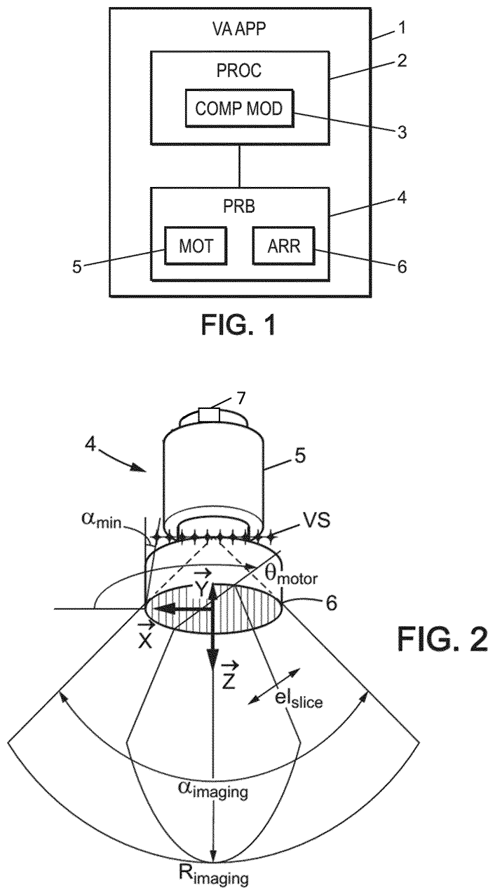

An example of apparatus 1 (VA APP) for imaging vascular activity usable in performing the method according to the present disclosure, is shown on FIG. 1.

The apparatus 1 may include a processor 2 (PROC), for instance a specialized signal processing device controlled by a computer or a group of computers, possibly a group of computers including servers.

The processor 2 may include a computing module 3 (COMP), the operation of which will be explained later.

The processor 2 may control a probe 4 (PRB).

The probe 4 may be for instance an ultrasonic probe in the example considered here.

The probe 4 may include an array 6 (ARR) of ultrasonic transducers. The array may be a linear array adapted to generate a 2D image of a slice of the region to be imaged, or a 2D array adapted to generate a 3D image of the region. When the array is a 2D array, it may be a sparse matrix of transducers, as known in the art.

Typical arrays of transducers may include a few hundreds to a few thousand of transducers. The array may also in some examples, be limited to one single transducer adapted to image only one line of the region, in the direction of the depth from the transducer or a few transducers adapted to image respectively lines of the region, in the direction of the depth from the transducer.

The following detailed description is done for the case of a linear or 2D array, so that the apparatus generates Doppler images (more generally: ultrasound measurements) having pixels (more generally: Doppler samples). In the case where the array would include just one transducer or a few transducers, the apparatus would generate an image limited to one line (ultrasound measurement) or a few lines in the direction of depth, the line(s) having pixels (the Doppler samples) and the process would be similar except for the generation of the ultrasound measurements which would not require inclined planar waves of different angles of inclination and would not require translated diverging waves from different virtual sources.

The transducer(s) may be adapted to transmit and receive ultrasound waves having a central frequency comprised for instance between 0.5 and 100 MHz, for instance between 1 and 20 MHz. One example of usable central frequency is 5 MHz.

In certain embodiments, the probe 4 may further include a motorization 5 (MOT) adapted to position the array 6.

An example of method of 3D Doppler scan acquisition will now be explained with regards to FIG. 2.

The array 6 of transducers may be controlled by processor 2 to transmit diverging ultrasonic waves in the region to be imaged and to receive the resulting backscattered ultrasonic waves, at a rate of for instance 6 kHz (Pulse Repetition Frequency PRF), i.e. around every 0.2 ms. More generally, the Pulse Repetition Frequency PRF may be over 500 Hz. The received signals are registered as a set of raw data for each transmitted diverging ultrasonic wave. The successive transmitted diverging waves are transmitted by N virtual sources (VS) which are regularly spaced with regards to the direction of the width (α_{imaging}) in the region to be imaged, i.e with regards to the direction lateral (\vec{x}) of the array 6. For instance, the position of the k^{th} virtual source (1≤k≤N) may defined by the following coordinates:

$$\{ \begin{matrix}{x}_{k} = ( L - z * tan ( {α}_{min} ) ) * ( - \frac{1}{2} + \frac{k - 1}{N - 1} ) \\ {y}_{k} = 0 \\ {z}_{k} = - sin ( \frac{{α}_{imaging}}{2} ) * L / 2\end{matrix}$$

where L is the probe's lateral aperture, for instance L=9.1 mm;
αₘᵢₙ is the minimum angle between virtual sources and extremity of the probe, for instance, αₘᵢₙ=10°;
α_{imaging} is the imaging width, for instance α_{imaging}=90°.

For each image of the region, a number N of diverging ultrasonic waves are successively transmitted by the N virtual sources (VS) and the N sets of raw data are processed to synthesize said image of the region, which is an In phase Quadrature (IQ) image reconstructed on a polar grid. The polar grid may be (elevation, radius)=(0.5°, 0.15 mm).

In the illustrated case of N=12 and PRF=6 KHz, the rate of the synthesized images of the region (framerate) is thus around 500 Hz. N may be different than 12, in which case the framerate of compound images is different. For instance, N=5 may be used.

Alternatively, the array 6 of transducers may be controlled by processor 2 to transmit planar ultrasonic waves in the region to be imaged and to receive the resulting backscattered ultrasonic waves, at a rate of for instance 5.5 kHz (Pulse Repetition Frequency PRF), i.e. around every 18 ms. More generally, the Pulse Repetition Frequency PRF may be over 500 Hz. The received signals are registered as a set of raw data for each transmitted planar ultrasonic wave. The successive transmitted planar waves have propagation direction which are inclined of varying successive angles with regards to the direction of the depth in the region to be imaged, i.e. with regards to the direction normal to the array 6. For each image of the region, a number N of planar ultrasonic waves are successively transmitted with different angles and the N sets of raw data are coherently added to synthesize said image of the region, which is thus a compound image. For instance, N may be 11 with angles varying between −10 deg and +10 deg by steps of 2 deg. In the case of N=11 and PRF=5.5 kHz, the rate of the compound images of the region (framerate) is thus 500 Hz. N may be different than 11, in which case the framerate of compound images is different. For instance N=5 may be used.

In both cases, based on the synthesized images of the region, Doppler images of the region are then computed by computing module 3. For instance, 500 successive synthesized images are used for each Doppler image. In this case, the rate of Doppler images is thus of 1 Hz. A different number of successive compound images may be used for each Doppler image, in which case the rate of Doppler images is different. For instance, 50 successive synthesized images could be used for each Doppler image, in which case the rate of Doppler images would be 10 Hz in the example considered here. Generally, the rate of Doppler images is at least 2 Hz.

The vascular parameter may be computed for instance from the successive synthesized images using spatiotemporal filtering based on single value decomposition (SVD). The successive synthesized images may be squared and averaged into a final ultrasensitive Doppler image. Owing to the high acquisition framerate of the images, it is possible to increase the sensitivity of the ultrasound signal without transmitting high energy ultrasound waves in the brain region.

More generally, the Doppler images may be computed by any Doppler technique, including ultra-sensitive Doppler, power Doppler, micro-Doppler, spectral Doppler. The Doppler signal constituting said Doppler images may be for instance power Doppler, color Doppler, spectral Doppler, index of vascular resistivity, or any combination thereof. Said Doppler signal may be filtered on different Doppler frequency bandwidths so as to assess sensitivity of the Doppler signal on blood velocity.

In the example of FIG. 2, the processor 2 rotates the array 6 to obtain a three-dimensional, 3D, Doppler scan of the region by acquiring Doppler images at different rotation angles. For instance, the array 6 is rotated over a 180° range by a 2° rotational step (θ_motor). That is, 89 Doppler images are used for each 3D Doppler scan. A different rotational step (θ_motor) may be used for each Doppler scan, in which case the spatial resolution is different. In particular, in some embodiments, the rotational step (θ_motor) may depend on the elevation (el_{slice}) of a Doppler image. For instance, the larger is the elevation of a Doppler image (el_{slice}), the smaller is the rotational step (θ_motor).

The 3D Doppler scans may be reconstructed from the Doppler images at different rotation angles by interpolating the Doppler images on a cartesian grid. The Doppler images may be corrected with a depth attenuation compensation. The (corrected) Doppler images may be interpolated in a polar grid with rotation (azimuth, elevation, radius)=(θₘₒₜₒᵣ, α_{imaging}, α_{imaging})=(2°, 0.5°, 0.15 mm) to cartesian grid (lateral, elevation, depth)=(x, y, z)=(0.25, 0.25, 0.25) mm³. Delaunay triangulation may be used for interpolating the (corrected) Doppler images.

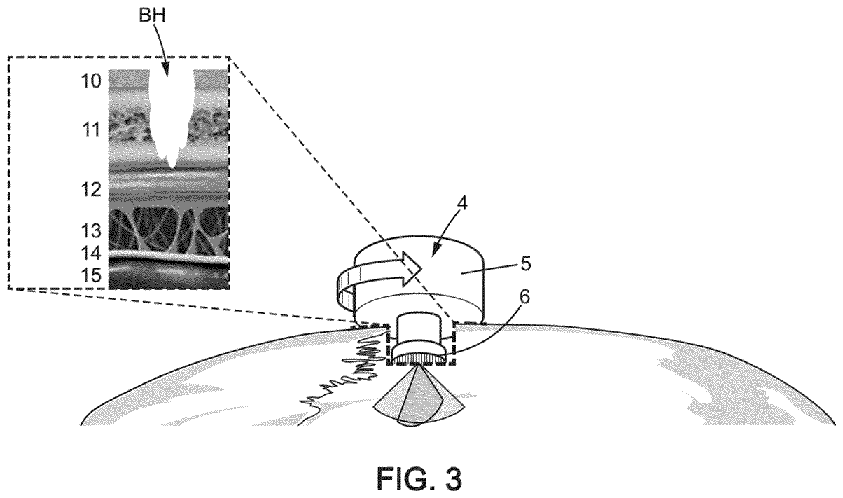

FIG. 3 schematically illustrates how the apparatus 1 may be used for imaging vascular activity in a region of the brain. The ultrasound probe 4 has been implanted through a burr hole (BH). The burr hole BH is a hole drilled in the skin 10 and the bone 11. The burr hole BH opens access to the brain 15 which is covered by the meninges. The meninges include inter alia three layers (dura mater 12, subarach 13, pia matter 14). The burr hole BH has a diameter which may be comprised between 10 mm and 20 mm, preferably around 15 mm.

In one embodiment, the method for implanting and imaging vascular activity in a region of a brain of patient comprises the following steps:
performing one burr hole into the skull of said human or animal;
implanting through the burr hole an implatable ultrasound probe;
obtaining a temporal series of three-dimensional, 3D, Doppler scans of said brain in said region based on ultrasound measurements of said region performed with said implantable ultrasound probe;
computing a temporal evolution of at least one vascular parameter of said region based on said 3D Doppler scans of said temporal series.

The surgery is performed under general anesthesia.

The position of the probe may be determined by the clinician based on the brain area to be monitored prior to drilling, e.g. by a brain-navigational system.

After skin antiseptisis, a 4 cm linear cutaneous incision on the skull can be made to expose the skull bone. A burr hole is made with a mechanical drill. The probe is positioned in the burr hole outside the dura mater which has to be preserved.

The implantable ultrasound probe is positioned through the burr hole and is fixed to the bone by any appropriate fixing means so that the implantable probe is secured with respect to the bone in the burr hole.

In one embodiment, the implantable ultrasound problem is connected to an analysis system which is external to the skull. For example, a connecting cable is tunneled under the skin for approximately 5 cm to emerge away from the first incision.

The skin incision is then sutured in 2 layers. Sterile dressings is made daily during the monitoring period.

At the end of the monitoring period, after skin antisepsis, the incision previously made is opened again. The means for fixing the probe to the bone is removed allowing removal of the probe and its cable. The incision is closed in 2 layers.

The ultrasound probe 4 is configured to fit in the burr hole BH. In the example of FIG. 3, the ultrasound probe 4 has a circular active face, whose diameter is smaller than the diameter of the burr hole BH. For instance, the diameter of the ultrasound probe 4 may be comprised between 6 mm and 16 mm, preferably 11 mm, depending on the diameter of the burr hole BH.

In some embodiments, the burr hole BH may have been previously drilled to help relieve pressure on the brain when brain tissue is compressed by fluid, such as flood, and/or to monitor intracranial pressure (ICP). Alternatively, the burr hole BH may be drilled to insert the ultrasound probe 4.

The motorization 5 rotates the array 6 within the burr hole BH to acquire the Doppler images of the 3D Doppler scans. The processor 2 is adapted to control the motorization 5.

In some embodiments, the processor 2 may be configured to obtain an additional temporal series of additional images of said brain in said region based on additional ultrasound measurements of said region performed with said ultrasound probe 4. An additional image may be acquired by the ultrasound probe 4 after each acquisition of a Doppler image. The additional image may be a B-mode ultrasound image and/or an elastography image.

The processor 2 may be configured to control the motorization 5 to observe a pause between two successive rotations of the array 6. For instance, a pause duration may above 8 s, preferably around 10 s. One example of usable pause duration is 11 s. In the exemplary case of a 11 s pause duration and with the acquisition of 89 Doppler images at a 1 Hz rate as described in the example of FIG. 2, a total duration for acquiring the 3D Doppler scans is hence approximately 18 min.

In variants, the ultrasound probe 4 may be further coupled to a temperature sensor (not shown). The computing module 3 may be adapted to select the pause duration, between two successive rotations, depending on a temperature measurement. The pause duration may be computed after each acquisition of a Doppler image such that an increase of temperature within the implanted ultrasound probe 4 does not exceed a threshold. The threshold may be set to 2 °C in accordance with the safety heating limit for implantable devices.

To achieve monitoring of the region, the processor 2 may be configured to control the probe 4 to continuously acquire 3D Doppler scans. That is, for two successive 3D Doppler scans of the temporal series, the acquisition of the last Doppler image of the former 3D Doppler scan may be separated from the acquisition of the first Doppler image of the latter 3D Doppler scan by the pause duration observed between two successive rotations of the ultrasound probe 4, i.e the pause duration observed between the acquisition of two successive Doppler images. Alternatively, the processor 2 may be configured to control the probe 4 to observe a pause between two successive 3D Doppler scans of the temporal series of a duration comprised for instance between 0 and 60 min, for instance between 1 and 20 min. One example of usable pause is 10 min. Such values allow to achieve both a high temporal resolution of the temporal series of 3D scans and an acceptable increase of temperature within the implanted ultrasound probe 4.

In a first embodiment, an angular position (θ_{last/former}) of the last Doppler image of the former 3D Doppler scan may correspond to θ_{last/former}=θ_{first/former}+180°−θₘₒₜₒᵣ, where θ_{first/former} corresponds to an angular position (θ_{first/former}) of the first Doppler image of the former 3D Doppler scan. An angular position (θ_{first/latter}) of the first Doppler image of the latter 3D Doppler scan may correspond to θ_{first/latter}=θ_{last/former}+θₘₒₜₒᵣ=θ_{first/former} mod (180°), where mod (180°) is the modulus. In this first embodiment, the array 6 may be rotated by incrementing the angular position by the rotation step (θ_motor). Alternatively, in a second embodiment, an angular position (θ_{first/latter}) of the first Doppler image of the latter 3D Doppler scan may correspond to θ_{first/latter}=θ_{last/former}−θₘₒₜₒᵣ. In this second embodiment, the array 6 may be rotated by decrementing the angular position by the rotation step (θ_motor). In some embodiments, the first and second embodiments may alternate in order to save one rotation of the array 6 between two successive 3D Doppler scans.

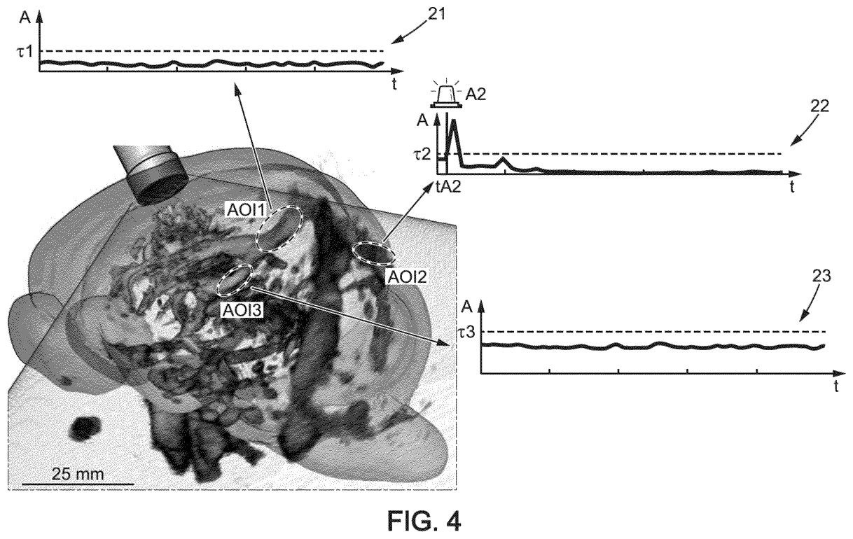

FIG. 4 illustrates a registration of a 3D Doppler scan on a known brain atlas, which is a preexisting cartography of the brain. Such maps are available for the human brain and some animal brains, e.g the swine. In some variants, the 3D Doppler scan may be alternatively or in combination registered to a CT scan of the subject's brain and/or a MR scan of the subject's brain.

Computing module 3 is adapted to select an area of interest of the 3D Doppler scan. The area of interest may be equal to the imaged region. Alternatively, the area of interest may be smaller than the imaged region. The areas of interest may be chosen based on vascular territories. For instance, the areas of interest may be chosen based on a vascular brain atlas. Alternatively or in combination, the areas of interest may be chosen based on a CT scan of the subject's brain and/or a MR scan of the subject's brain. Depending on the embodiments, the areas of interest may or may not cover the entire imaged region.

Once the area of interest is determined, the computing module 3 may average the Doppler signal on the area of interest through the 3D Doppler scans, thus obtaining the temporal evolution of the vascular parameter in the area of interest. Additionally, when additional temporal series are acquired, the B-mode ultrasound signal and/or the elastography signal may be average on the area of interest through the additional images, thus obtaining the temporal evolution of the additional vascular parameter in the area of interest. What is being explained below for the vascular parameters based on the 3D Doppler scans is applicable also for the additional vascular parameters, except for the features which are specific to the Doppler modality.

In the example of FIG. 4, three areas of interest (AOI1, AOI2, AOI3), and the associated curves of temporal evolution (21, 22, 23), with time (T) in abscissa and the value of the vascular parameter (VP) in ordinate, are represented.

The represented areas of interest AOI1, AOI2, AOI3 correspond respectively to the Internal Cerebral Vein (ICV), a Transverse Sinus (TS) and a Thalamostriate System vein (TsS).

The temporal evolution of the vascular parameter in the area of interest may be used as a reliable biomarker of certain health disorders related to brain vascular activity, such as delayed ischemia, vasospasm, hemorrhage, hypoperfusion, hyperperfusion or edema. For instance, a decrease in the vascular parameter's values in the area of interest may correspond to an ischemia in the area of interest.

To this end, computing module 3 may be adapted to compute at least one vascular parameter from the temporal series of 3D Doppler scans, said at least one parameter being chosen in the group comprising blood flow, blood velocity, in particular systolic velocity and/or diastolic velocity, blood volume and arterial resistivity index.

In some embodiments, relative 3D Doppler scans are computed. The at least one vascular parameter may hence be a difference between values observed in two 3D Doppler scans.

For determining whether the temporal evolution of the vascular parameter is normal, computing module 3 may compare said evolution to a threshold. The threshold may be set by an expert, such as a medical practitioner.

In a variant, a baseline of the vascular parameter is obtained and the threshold is based on the baseline. For instance, the threshold may be a percentage of the baseline. The percentage may be comprised between −100% and +500%, preferably −50%. For example, a percentage comprised between −100% and −10%, preferably −50% may be chosen when the vascular parameter is the blood volume. Hence, the threshold may allow to detect a decrease in the blood volume, possibly due to an ischemia. In another example, a percentage comprised between +10% and +500%, preferably +50% may be chosen when the vascular parameter is the blood flow. Hence, the threshold may allow to detect an increase in the blood flow, possibly due to an incipient vasospasm.

In a variant, the baseline is derived from the first 3D Doppler scan of the temporal series of 3D Doppler scans. For instance, the baseline is the value of the vascular parameter in the 3D Doppler scan of the temporal series of 3D Doppler scans.

Alternatively or in combination, in a variant, an additional baseline is derived from a first Ultrasound Localization Microscopy (ULM) image of said brain in said region. For instance, additional the baseline is the value of the vascular parameter in the first ULM image. The first ULM image may be a 2D or a 3D image of said brain in said region, preferably a 3D image of said brain in said region. The first ULM image used for the additional baseline may be obtained prior to the acquisition of the temporal series of 3D Doppler scans. The first ULM image used for the additional baseline and the 3D Doppler scans may be obtained using the same probe 4. The first ULM image used for the additional baseline may be obtained using the same parameters as for the 3D Doppler scans. In particular, the same rotational step θ_motor may be used.

In another variant, the threshold depends on the diameter of the vessel in the area of interest. For instance, the threshold may be ponderated by a percentage of the vessel's diameter. In another variant, the threshold depends on the elasticity of the vessel in the area of interest. For instance, the threshold may be ponderated by a percentage of the vessel's elasticity. These variants may be implemented, separately or in combination one with the others.

In the example of FIG. 4, different thresholds (τ₁, τ₂, τ₃) are set for the different areas of interest. An alert A2 is triggered based on the vascular parameter in the area of interest AOI2 exceeding the threshold τ₂ at time tA2.

The processor 2 may further be configured to trigger the alert based on a duration of the variation of the temporal evolution of the at least one vascular parameter exceeding the threshold. For instance, the alert may be triggered when the variation of the temporal evolution of the at least one vascular parameter exceeds the threshold for a duration comprised between 1 s and 10 h, preferably 1 min. Therefore, an isolated instaneous variation of the temporal evolution exceeding the threshold may not be construed as an anormal variation of the vascular parameter.

The apparatus 1 as described above may also include an accelerometer 7 coupled to the ultrasound probe 4. The accelerometer may detect a movement of the human or animal whose brain is being imaged. The alert may be triggered based on said detection of said movement. For instance, when the accelerometer detects a movement of said human or animal due to an external cause, such as a movement/manipulation of the body, the alert may not be triggered even if the vascular parameter exceeds the threshold. In particular, in this case, the alert may be triggered only if the vascular parameter exceeds the threshold for a predetermined duration, as explained above.

The apparatus 1 may further be configured to obtain an additional second ultrasound localization microscopy image of the brain in the area of interest for which an alert has been triggered. The additional second image may be a 2D image or a 3D image.

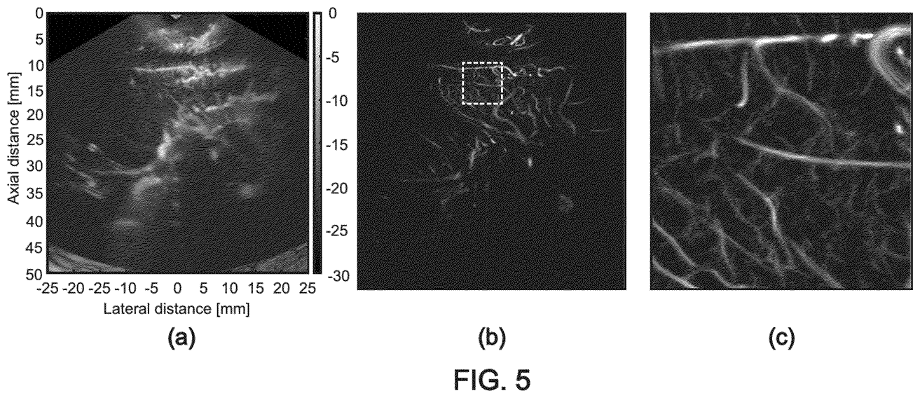

In the example of FIG. 5(a), a 2D Doppler image of a region of the brain has been obtained with the method described in reference to FIG. 2. The additional second image of FIG. 5(b) is an Ultrasound Localization Microscopy (ULM) image of the same region of the brain. FIG. 5(c) is a zoom of the dashed subregion of FIG. 5(b). The second ULM images of FIGS. 5(b) and 5(c) reach subwavelength resolution. The second ULM images of FIGS. 5(b) and 5(c) may be used to precisely quantify the vascular parameter. In particular, the blood flow velocity and direction may be precisely quantified.

In a variant, when an additional baseline is derived from the first ULM image of said brain in said region, the second ULM image of said brain in said region may be compared with the first ULM image and/or the additional baseline.

In particular, in a variant, in a first step, the additional baseline is derived from the first ULM image of said brain in said region. In a second step, the temporal series of 3D Doppler scans is obtained and the temporal evolution of the at least one vascular parameter is computed with a method of the disclosure. As described above, an alert may be triggered based on a variation of the temporal evolution of the at least one vascular parameter exceeding a threshold. The alert may depend on a baseline of the at least one vascular parameter. Said baseline may have been obtained based on a 3D Doppler scan, for instance the first 3D Doppler scan of the temporal series. Based on said alert, at least a second ULM image of said brain in said region may be obtained with the same probe 4 as for the first ULM image and the 3D Doppler scans of the temporal series. The second ULM image may be compared with the first ULM image and/or the additional baseline derived from the first ULM image. That is, the temporal series of 3D Doppler scans and the resulting temporal evolution of the at least one vascular parameter may allow to monitor the brain in the region, to detect an anormal activity and to trigger an alert. Thanks to the first ULM image acquired prior to the temporal series of 3D Doppler scan and to the second ULM image acquired upon triggering of the alert, it may be possible to quantify the change in the vascular activity of the region with a microscopic resolution. Hence, a continuous monitoring of the vascular activity, through the 3D Doppler scans, and a precise quantification of the change in the vascular activity upon detection of an alert, through the ULM images, may be achieved with a method of the disclosure.

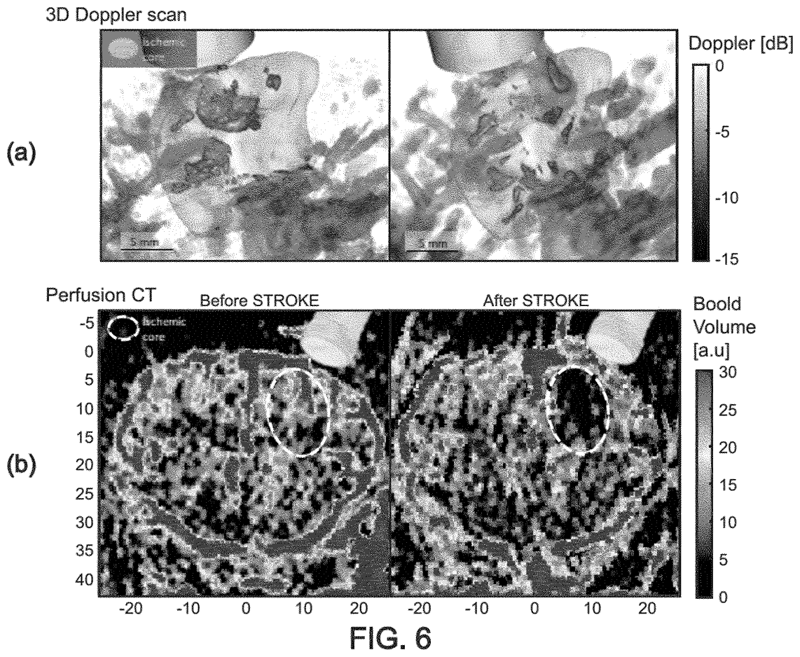

As shown on FIG. 6(a), stroke detection following an ischemia may be achieved with a method of the disclosure. In particular, in the example of FIG. 6(a), fewer vessels are observed in the outlined ischemic core of the 3D Doppler scan after stroke (on the right of FIG. 6(a)) than in the outlined ischemic core of the 3D Doppler scan before stroke (on the left of FIG. 6(b)). Further, a decreased of 25% (0.161 to 0.105 normalized Doppler signal) of the Doppler signal in the ischemic core is observed between the 3D Doppler scan before stroke and the 3D Doppler scan after stroke.

A computed tomography (CT) imaging experiment, shown on FIG. 6(b), confirms the formation of the ischemic core after the stroke. The perfusion computed tomography scans of FIG. 6(b) exhibit blood volumes on a sagittal view. A perfusion dropping by around 60% (14.0 to 4.9 a.u. CT's blood volume) is observed in the dashed region of the ischemic core between the CT scan acquired before stroke (on the left of FIG. 6(b)) and the CT scan acquired after stroke (on the right of FIG. 6(b)).

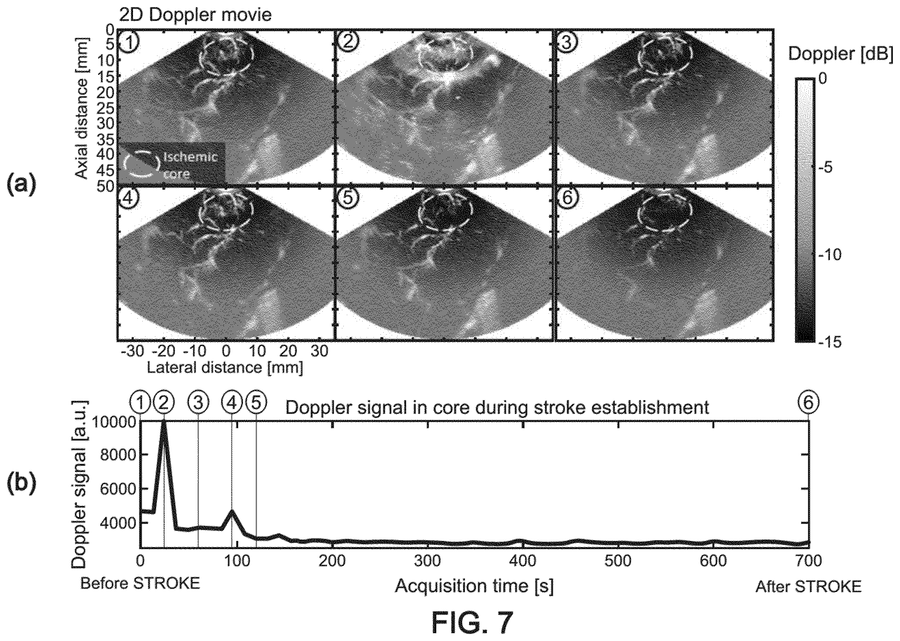

FIG. 7(a) shows 2D Doppler images of the same region of the brain acquired during the establishment of the stroke assessed in FIGS. 6(a) and 6(b). The 2D Doppler images are acquired with a method of the disclosure. For instance, based on the temporal series of 3D Doppler scans on which the ischemic core is observed, the 2D Doppler images (based on which the 3D Doppler scans were constructed) corresponding to the region of the ischemic core at different times may be retrieved.

FIG. 7(b) shows an example of a measure of an evolution of the Doppler signal in the ischemic core along acquisition of 2D Doppler images including the 2D Doppler images of FIG. 7(a). A large Doppler spike is detected in FIG. 7(a)(2), which succeeds a baseline acquired in FIG. 7(a)(1) and precedes a significant decrease (blood flow is diminished by about 38%) observed in FIG. 7(a)(3) to FIG. 7(a)(5). The large Doppler spike and following significant decrease may probably be due to a clot migrating preceding the blockage of a vessel. Hence, the measure of the evolution of the Doppler signal may be used as a reliable biomarker of ischemic core formation.

What has been explained above may be duplicated when the subject has two or more burr holes. In this case, the computed module 3 may control two or more ultrasound probes 4, which have been inserted in the two or more burr holes respectively, in parallel, to implement at least part of a method previously described. Larger regions of the brain may hence be imaged. Further, information acquired by the two or more ultrasound probes 4 may be gathered to achieve a higher spatial resolution.

Notably, the apparatus 1 as described above may also be used to monitor brain perfusion during surgeries. In particular, the apparatus 1 may be used to provide a reliable measure of cerebral blood flow across the whole brain during long surgeries, such as heart surgeries, which may last for hours. Hence, early detection of vascular accidents during surgeries may be achieved.

1. A method for imaging vascular activity in at least one region of a brain, of a human or animal, comprising:
(a) obtaining a temporal series of three-dimensional, 3D, Doppler scans of said brain in said region based on ultrasound measurements of said region performed with an ultrasound probe having an array of at least one ultrasound transducer, the ultrasound probe having been implanted through a burr hole in said human or animal; and
(b) computing a temporal evolution of at least one vascular parameter of said region based on said 3D Doppler scans of said temporal series.

2. The method according to claim 1, further comprising:
(a1) obtaining an additional temporal series of additional images of said brain in said region based on additional ultrasound measurements of said region performed with said ultrasound probe; and
(b1) computing an additional temporal evolution of at least one vascular parameter of said region based on said additional images of said additional temporal series, wherein the additional images are chosen in the group comprising: B-mode ultrasound images, elastography images and any combination thereof.

3. The method according to claim 1, further comprising:
(c) triggering an alert (A2) based on a variation of the temporal evolution of the at least one vascular parameter exceeding a threshold.

4. The method according to claim 3, further comprising obtaining a baseline of the at least one vascular parameter and wherein computation of the variation of the temporal evolution of the at least one vascular parameter depends on the baseline.

5. The method according to claim 1, further comprising:
(d) obtaining at least a first ultrasound localization microscopy image of said brain in said region based on first additional ultrasound measurements of said region performed with said ultrasound probe.

6. The method according to claim 5 further comprising triggering an alert (A2) based on a variation of the temporal evolution of the at least one vascular parameter exceeding a threshold, wherein based on the triggered alert (A2), the method further includes:
(e) obtaining at least a second ultrasound localization microscopy image of said brain in said region based on second additional ultrasound measurements of said region performed with said ultrasound probe,
(f) comparing said at least second ultrasound localization microscopy image with said at least first ultrasound localization microscopy image.

7. The method according to claim 1, wherein the at least one vascular parameter is chosen in the group comprising: blood flow, blood velocity, blood volume, arterial resistivity index and any combination thereof.

8. The method according to claim 1, wherein at least one 3D Doppler scan of said temporal series is registered to at least one of a brain atlas, a computed tomography scan of said brain, a magnetic resonance image scan of said brain and any combination thereof.

9. The method according to claim 1, wherein said temporal series of 3D Doppler scans of said brain in said region is divided into areas of interest, and wherein the method further includes for each area of interest:
(b1) computing a temporal evolution of at least one vascular parameter of said area of interest based on said 3D Doppler scans of said temporal series.

10. The method according to claim 2, wherein the ultrasound probe is coupled to an accelerometer configured to detect a movement of said human or animal and wherein an alert is triggered based on said detection of said movement.

11. The method according to claim 1, wherein the ultrasound probe is rotated between measurements to obtain at least a 3D Doppler scan of said temporal series.

12. The method according to claim 11, wherein a pause is observed between two successive rotations of the ultrasound probe.

13. An apparatus for imaging vascular activity in at least one region of a brain, of a human or animal, comprising:
(a) an ultrasound measuring device adapted to perform ultrasound measurements of said region with an ultrasound probe having an array of at least one ultrasound transducer, the ultrasound probe being implantable through a burr hole (BH) in said human or animal; and
(b) a computing module adapted to:
compute, from said ultrasound measurements, a temporal series of three-dimensional, 3D, Doppler scans of said region; and
compute a temporal evolution of at least one vascular parameter of said region based on said 3D Doppler scans of said temporal series.

14. The apparatus according to claim 13, wherein the computing module (3) is adapted to detect a seizure and/or a delayed ischemia, vasospasm, hemorrhage, hypoperfusion, hyperperfusion or edema based on the temporal evolution of the at least one vascular parameter of said region.

15. Computer software encoded on a non-transient storage medium comprising instructions to implement at least a part of a method according to claim 1 when the software is executed by a processor.

16. Computer-readable non-transient recording medium on which a software is registered to implement a method according to claim 1 when the software is executed by a processor.
